(19) United States
(12) Patent Application Publication
(10) Pub. No.: US 2004/0019980 A1
(43) Pub. Date: Feb. 5, 2004

(54) One step hair coloring compositions using salts

(75) Inventors: Van Au, New City, NY (US); Stephen Alan Madison, New City, NY (US); John Brian Bartolone, Bridgeport, CT (US); Gabriela Wis-Surel, Cos Cob, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/211,909

(22) Filed: Aug. 2, 2002

(51) Int. Cl. A61K 7/13

(52) U.S. Cl. 8/405; 8/406; 8/408

(57) ABSTRACT

A hair coloring composition comprising the following two compositions which are mixed just prior to application to the hair:

(a) a composition comprising a water-soluble peroxygen oxidizing agent; and

(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises al least one water soluble carbonate releasing salts; and optionally a water soluble ammonium salt,

is described.

### BACKGROUND OF THE INVENTION

To color human or animal hair using conventional oxidative dye technology it is generally necessary to treat the hair with a mixture of suitable oxidative coloring agents and at least one oxidizing agent and swelling agent at alkaline pHs. Hydrogen peroxide is the most commonly used oxidizing agent. However, there is a recognized need to develop hair care compositions which more effectively deliver hair color. It is a purpose of the present invention to provide such a hair coloring composition.

The following publications relate to the field of the invention:

U.S. Pat. No. 5,131,912 discloses durable 2-part hair dyeing agents composed of a first agent comprising as essential components at least one compound that forms HCO₃—by dissociation in water, an alkali generating substantially no irritating odor and a dye for hair and having a pH of 8.2 to 9.0. and a second agent comprising as essential components hydrogen peroxide and a buffer solution and having a pH of 2.0 to 4.0, the weight ratio of the first agent and the second agent to be mixed being such that the pH of the mixture of the two is in a range of from 6.5 to 7.9. These 2-part hair dyeing agents require only a short dyeing time, create little damage to hair and no irritating or disagreeable odor and have high dyeing effect.

U.S. Pat. No. 5,525,123, discloses a hair dyeing composition based on oxidation dyestuff precursors which dyes and brightens the hair containing, besides at least one developing and at least one coupling agent, at least one metal salt and at least one ammonium compound selected from the group ammonium chloride, ammonium sulfate, ammonium carbonate, ammonium bicarbonate, and ammonium carbamate, having a pH-value between 8 and 11, preferably from 9 to 10, after admixture with an oxidizing agent in the ready-to-use preparation.

### SUMMARY OF THE INVENTION

The present invention is related to a hair coloring composition suitable for the treatment of human or animal hair. According to one aspect of the present invention, there is provided a hair coloring composition comprising the following two compositions which are mixed just prior to application to the hair:

(a) a composition comprising a water-soluble peroxygen oxidizing agent; and

(b) a composition comprising one or more oxidative hair coloring agents selected from the group consisting of an aromatic diamine, an amino phenol, a naphthol, a polyhydric phenol, a catechol and mixtures thereof: wherein the composition comprising one or more oxidative hair coloring agents further comprises at least one water soluble carbonate releasing salt; and optionally a water soluble ammonium salt.

### DETAILED DESCRIPTION OF THE INVENTION

As used herein, % means weight % of the total composition unless otherwise designated. The compositions of the invention are made using known ingredients or with ingredients analogous to those known in the art. The packages or containers to be used with the compositions of the invention are made using known processes and materials or by processes and materials which are analogous to those known in the art.

Compositions of the invention are used in a one step process for the coloring and bleaching of hair. That is, the composition is made by mixing the following two ingredients just prior to application to the hair;

(a) a composition comprising a water-soluble peroxygen oxidizing agent; and

(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises al least one water soluble carbonate releasing salts; and optionally a water soluble ammonium salt.

After mixture, this composition is then applied to hair. This composition is allowed to remain on the hair for about 2 minutes to about 60 minutes. The coloring reaction takes place and the hair is rinsed.

As used herein the term “hair” to be treated may be “living” i.e. on a living body or may be “non-living” i.e. in a wig, hairpiece or other aggregation of non-living fibres. Mammalian, preferably human hair is preferred. However wool, fur and other melanin containing fibres are suitable substrates for the compositions according to the present invention.

The hair coloring compositions can contain, in addition to a mixture of active oxidizing agents and oxidative coloring agents, ingredients such as, by way of example, sequestrants, thickeners, buffers, carriers, surfactants, solvents, antioxidants. polymers, non-oxidative dyes and conditioners.

As noted above, there is provided a hair coloring composition which comprises the following two compositions which are mixed just prior to application to the hair:

(a) a composition comprising a water-soluble peroxygen oxidizing agent; and

(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises at least one water soluble carbonate releasing salt; and optionally a water soluble ammonium salt.

The water-soluble peroxygen oxidizing agent may be selected from hydrogen peroxide, sodium perborate, sodium percarbonate, and urea peroxide complexes.

The composition comprising a water-soluble peroxygen oxidizing agent may further comprises a buffering agent.

The mixture of the composition comprising water-soluble peroxygen oxidizing agent and the composition having one or more oxidative hair coloring agents as described above has a pH of about 8 to about 11.

The composition having one or more oxidative hair coloring agents and the composition comprising a water-soluble peroxygen oxidizing agent may further comprises a surfactant selected from the group consisting of anionic, nonionic, cationic, zwitterionic, amphoteric surfactants and mixtures thereof.

The invention also relates to a hair coloring kit comprising an individually packaged oxidizing component with additional agents, an individually packaged component having one or more oxidative hair coloring agents which when mixed forms a composition of the invention.

The invention also relates to a process for coloring human or animal hair which comprises applying to the hair the compositions described above.

Change in hair color can be measured by means which are known in the art. A customary scale for measuring the change in hair color is defined in terms of the variables: L, a, and b wherein L, a, and b are defined as follows: L indicates the lightness or darkness of the color value. The higher the L, therefore, the lighter the hair, and the more fading that has occurred. When L is 0, the hair is black, and when L is 100, the hair is white. −a and +a represent changes in color tone from green to red. −b and +b represent the changes in color tone from blue to yellow.

What follows now is a description of the ingredients which may be used in the compositions of the invention.

Water-Soluble Peroxygen Oxidizing Agents

The compositions of the invention may comprise at least one water-soluble peroxygen oxidizing agent. Water-soluble as defined herein means a peroxygen oxidizing agent compound, which can be substantially solubilized in water.

The peroxygen oxidizing agents useful herein are generally inorganic peroxygen materials capable of yielding hydrogen peroxide in an aqueous solution. Water-soluble peroxygen oxidizing compounds are well known in the art and include hydrogen peroxide, inorganic alkali metal peroxides such as sodium periodate and sodium peroxide and organic peroxides such as urea peroxide, melamine peroxide, and inorganic perhydrate salt oxidizing compounds, such as the alkali metal salts of perborates, percarbonates, perphosphates, persilicates, persulphates and the like. These inorganic perhydrate salts may be incorporated as monohydrates, tetrahydrates, and the like. Mixtures of two or more such oxidizing agents can be used if desired. Preferred for use in the compositions according to the present invention is hydrogen peroxide.

Oxidative Hair Coloring Agents

The compositions of the present invention may include one or more oxidative hair coloring agents. Such oxidative hair coloring agents are used in combination with the oxidizing systems of the present invention to formulate permanent, hair dye compositions.

Permanent hair dye compositions as described herein are compositions, which once applied to the hair are substantially resistant to wash-out. Wash-out as described herein is the process by which hair color is removed from the hair over time during normal hair cleansing regimen.

When compared against a combination of all of the compositions of the invention after they have been mixed together, the concentration of each oxidative hair coloring agent is from about 0.0001% to about 7% by weight and is preferably from about 0.001% to about 2.0% by weight.

When compared against a combination of all of the compositions of the invention after they have been mixed together, the total combined level of oxidative hair coloring agents in the compositions according to the present invention is from about 0.01% to about 15%, preferably from about 0.01% to about 10%, more preferably from about 0.1% to about 5% by weight.

Oxidative hair coloring agents which can be used in compositions of the invention can be selected from the group consisting of an aromatic diamine, an aminophenol, a polyhydric phenol, a naphthol, a catechol and mixtures thereof. Oxidative hair coloring agents which can also be called oxidative dyes are described in more detail below.

The dye forming intermediates used in oxidative dyes can be aromatic diamines, aminophenols and their derivatives. These dye-forming intermediates can be classified as; primary and secondary intermediates. Primary intermediates are chemical compounds, which by themselves will form a dye upon oxidation. The secondary intermediates, are also known as color modifiers or couplers and are used with other intermediates for specific color effects or to stabilize the color.

The oxidation dye intermediates which are suitable for use in the compositions and processes herein include aromatic diamines, polyhydric phenols, aminophenols and derivatives of these aromatic compounds (e.g., N-substituted derivatives of the amines, and ethers of the phenols). Primary oxidation dye intermediates are generally colorless molecules prior to oxidation. The oxidation dye color is generated when the primary intermediate is activated and subsequently joined with a secondary intermediate (coupling agent), which is also generally colorless, to form a colored, conjugated molecule.

In general terms, oxidation hair dye precursors or intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear colored. For example, oxidation dye precursors capable of forming colored polymers include materials such as aniline, which has a single functional group and which, on oxidation, forms a series of conjugated imines and quinoid dimers, trimers, and the like, ranging in color from green to black. Compounds such as p-phenylenediamine, which has two functional groups, are capable of oxidative polymerization to yield higher molecular weight colored materials having extended conjugated electron systems. Color modifiers (couplers), such as those detailed hereinafter, are preferably used in conjunction with the oxidation dye precursors herein. A representative list of oxidation dye precursors suitable for use is found in Sagarin, “Cosmetic Science and Technology Interscience, Special Edn. Vol 2 pages 308 to 310, which is hereby incorporated by reference.

The typical aromatic diamines, polyhydric phenols, naphthols, aminophenols, and derivatives thereof, described above as primary dye precursors can also have additional substituents on the aromatic ring, e.g. halogen, aldehyde, carboxylic acid, nitro, sulfonic acid and substituted and unsubstituted hydrocarbon groups, as well as additional substituents on the amino nitrogen and on the phenolic oxygen, e.g. substituted and unsubstituted alkyl and aryl groups.

Nonlimiting examples of suitable aromatic diamines, aminophenols, naphthols, polyhydric phenols and derivatives thereof, respectively, are the following compounds:

o-phenylenediamine,

m-phenylenediamine,

2-nitro-p-phenylenediamine,

1,3,5-triaminobenzene,

2-hydroxy-p-phenylenediamine,

2,4-diaminobenzoic acid,

sodium 2,4-diaminobenzoate,

calcium di-(2,4-aminobenzoate),

ammonium 2,4-diaminobenzoate,

trimethylammonium 2,4-diaminobenzoate,

tri-(2-hydroxyethyl)ammonium 2,4-diaminobenzoate,

2,4-diaminobenzaldehyde carbonate,

2,4-diaminobenzenesulfonic acid,

potassium 2,4-diaminobenzenesulfonate,

N,N-diisopropyl-p-phenylenediamine bicarbonate,

N,N-dimethyl-p-phenylenediamine,

N-ethyl-N′-(2-propenyl)-p-phenylenediamine,

N-phenyl-p-phenylenediamine,

N-phenyl-N-benzyl-p-phenylenediamine,

N-ethyl-N′-(3-ethylphenyl)-p-phenylenediamine,

2,4-toluenediamine,

2-ethyl-p-phenylenediamine,

2-(2-bromoethyl)-p-phenylenediamine,

2-phenyl-p-phenylenediamine laurate,

4-(2,5-diaminophenyl)benzaldehyde,

2-benzyl-p-phenylenediamine acetate,

2-(4-nitrobenzyl)-p-phenylenediamine,

2-(4-methylphenyl)-p-phenylenediamine,

2-(2,5-diaminophenyl)-5-methylbenzoic acid,

2-methoxy-p-phenylenediamine,

2,3-dimethyl-p-phenylenediamine,

2,5-dimethyl-p-phenylenediamine,

2-methyl-5-methoxy-p-phenylenediamine,

2,6-methyl-5-methoxy-p-phenylenediamine,

3-methyl-4-amino-N,N-diethylaniline,

N,N-bis-(2-hydroxyethyl)-p-phenylenediamine,

3-methyl-4-amino-N,N-bis-(2-hydroxyethyl)aniline,

3-chloro-4-amino-N,N-bis-(2-hydroxyethyl)aniline,

4-amino-N-ethyl-(piperidonoethyl)aniline,

3-methyl-4-amino-N-ethyl-β-(piperidonoethyl)aniline,

4-amino-N-ethyl-N-(morpholinoethyl)aniline,

4-amino-N-ethyl-N-(acetylaminoethyl)aniline,

4-amino-N-(methoxyethyl)aniline,

3-methyl-amino-N-ethyl-N-(2-acetylaminoethyl)aniline,

4-amino-N-ethyl-N-(mesylaminoethyl)aniline,

3-methyl-4-amino-N-ethyl-N-(β-mesylaminoethyl)aniline,

4-amino-N-ethyl-N-(β-sulfoethyl)aniline,

3-methyl-4-amino-N-ethyl-N-(β-sulfoethyl)aniline,

N-(4-aminophenyl)morpholine,

N-(4-aminophenyl)piperidine,

2,3-dimethyl-p-phenylenediamine,

2-isopropyl-p-phenylenediamine,

N,N-bis-(2-hydroxyethyl)-p-phenylenediamine sulfate,

o-aminophenol,

m-aminophenol,

p-aminophenol,

2-iodo-p-aminophenol,

2-nitro-p-aminophenol,

3,4-dihydroxyaniline,

3,4-diaminophenol,

2-hydroxy-4-aminobenzoic acid,

2-hydroxy-4-aminobenzaldehyde,

3-amino-4-hydroxybenzenesulfonic acid,

N,N-diisopropyl-p-aminophenol,

N-methyl-N-(1-propenyl)-aminophenol,

N-phenyl-N-benzyl-p-aminophenol sulfate,

N-methyl-N-(3-ethylphenyl)-p-aminophenol,

2-nitro-5-ethyl-p-aminophenol,

2-nitro-5-(2-bromoethyl)-p-aminophenol,

(2-hydroxy-5-aminophenyl)acetaldehyde,

2-methyl-p-aminophenol,

(2-hydroxy-5-aminophenyl)acetic acid,

3-(2-hydroxy-5-aminophenyl-1-propene,

3-(2-hydroxy-5-aminophenyl)-2-chloro-1-propene.

2-phenyl-p-aminophenol palmitate,

2-(4-nitrophenyl)-p-aminophenol,

2-benzyl-p-aminophenol,

2-(4-chlorobenzyl-p-aminophenol perchlorate,

2-(4-methylphenyl)-p-aminophenol,

2-(2-amino-4-methylphenyl)-p-aminophenol,

p-methoxyaniline,

di-(2-aminoethyl-4-aminophenyl)ether,

di-(2-hydroxyethyl-4-aminophenyl)ether,

(4-aminophenoxy)acetaldehyde,

(4-aminophenoxy)acetic acid,

(4-aminophenoxy)methanesulfonic acid,

1-propenyl-4-aminophenyl ether isobutyrate,

di-(2-chloro-1-propenyl-4-aminophenyl)ether,

di-(2-nitro-1-propenyl-4-aminophenyl)ether,

di-(2-amino-propenyl-4-aminophenyl)ether,

di-(2-hydroxy-1-propenyl-4-aminophenyl)ether,

N-methyl-p-aminophenol,

3-methyl-4-aminophenol,

2-chloro-4-aminophenol,

3-chloro-4-aminophenol,

2,6-dimethyl-4-aminophenol,

3,5-dimethyl-4-aminophenol,

2,3-dimethyl-4-aminophenol,

2,5-dimethyl-4-aminophenol,

2-hydroxymethyl-4-aminophenol,

3-hydroxymethyl-4 aminophenol,

o-hydroxyphenol (catechol),

m-hydroxyphenol (resorcinol),

p-hydroxyphenol (hydroquinone),

4-methoxyphenol,

2-methoxyphenol,

4-(2-chloroethoxy)phenol,

4-(2-propenoxy)phenol,

4-(3-chloro-2-propenoxy)phenol,

2-chloro-4-hydroxyphenol (2-chlorohydroquinone),

2-nitro-hydroxyphenol (2-nitrohydroquinone),

2-amino-4-hydroxyphenol,

1,2,3-trihydroxybenzene (pyrogallol),

2,4-dihydroxybenzaldehyde,

3,4-dihydoxybenzoic acid,

2,4-dihydroxybenzenesulfonic acid,

3-ethyl-4-hydroxyphenol,

3-(2-nitroethyl)-4-hydroxyphenol,

3-(2-propenyl)-4-hydroxyphenol,

3-(3-chloro-2-propenyl)-4-hydroxyphenol,

2-phenyl-4-hydroxyphenol,

2-(4-chlorophenyl)-4-hydroxyphenol,

2-benzyl-4-hydroxyphenol,

2-(2-nitrophenyl)-4-hydroxyphenol,

2-(2-methylphenyl)-4-hydroxyphenol,

2-(2-methyl-4-chlorophenyl)-4-hydroxyphenol,

2-methoxy-4-(1-propenyl)phenol,

4-hydroxy-3-methoxycinnamic acid,

2,5-dimethoxyaniline.

2-methylresorcinol.

aniline,

p-chloroaniline,

p-fluoroaniline,

p-nitroaniline,

p-aminobenzaldehyde,

p-aminobenzoic acid,

sodium p-aminobenzoate,

lithium p-aminobenzoate,

calcium di-(p-aminobenzoate),

ammonium p-aminobenzoate,

p-aminobenzenesulfonic acid,

potassium p-aminobenzenesulfonate,

N-methylaniline,

N-propyl-N-phenylaniline,

N-methyl-N-2-propenylaniline,

N-benzylaniline,

N-(2-ethylphenyl)aniline,

4-methylaniline,

4-(2-bromoethyl)aniline,

2-(2-nitroethyl)aniline,

4-aminophenylacetaldehyde,

4-aminophenylacetic acid,

4-(2-propenyl)aniline acetate,

4-(3-bromo-2-propenyl)aniline,

4-phenylaniline chloroacetate,

4-(3-chlorophenyl)aniline,

4-benzylaniline,

4-(4-iodobenzyl)aniline,

4-(3-ethylphenyl)aniline,

4-(2-chloro-ethylphenyl)aniline,

phenol,

p-chlorophenol,

p-nitrophenol,

p-hydroxybenzaldehyde,

p-hydroxybenzoic acid,

p-hydroxybenzenesulfonic acid,

ethylphenyl ether,

di-(2-chloroethylphenyl)ether

di-(2-nitroethylphenyl)ether,

phenoxyacetaldehyde,

phenoxyacetic acid,

3-phenoxy-1-propene,

3-phenoxy-2-nitro-1-propene,

3-phenoxy-2-bromo-1-propene,

4-propylphenol,

4-(3-bromopropyl)phenol,

2-(2-nitroethyl)phenol,

4-hydroxyphenylacetaldehyde,

4-hydroxyphenylacetic acid,

4-(2-propenyl)phenol,

4-phenylphenol

4-benzylphenol,

4-(3-fluoro-2-propenyl)phenol,

4-(4-chlorobenzyl)phenol,

4-(3-ethylphenyl)phenol

4-(2-chloro-3-ethylphenyl)phenol,

2,5-xylenol,

2,5-diaminopyridine,

2-hydroxy-5-aminopyridine,

2-amino-3-hydroxypyridine.

tetraaminopyrimidine,

1,2,4-trihydroxybenzene

1,2,4-trihydroxy-5-(C₁-C₆-alkyl)benzene,

1,2,3-trihydroxybenzene,

4-aminoresorcinol,

1,2-dihydroxybenzene,

2-amino-1,4-dihydroxybenzene,

2-amino-4-methoxyphenol,

2,4-diaminophenol,

3-methoxy-1,2-dihydroxybenzene,

4,6-dimethoxy-3-amino-1-hydroxybenzene,

2,6-dimethyl-4-(p-hydroxyphenyl)amino]phenol;

4-benzylnaphth-1-ol,

4-chloronaphth-1-ol,

4-chloro-5,8-dimethoxy-6-methyinaphth-1-ol,

4-chloro-5,8-dimethoxynaphth-1-ol,

4-acetoxy-5-chloro-6-methyl-7-acetyl-8-hydroxynaphth-1-ol,

4-acetoxy-6-methyl-7-acetyl-8-hydroxynaphth-1-ol,

4-acetoxy-8-benzyloxynaphth-1-ol,

4-benzyloxynaphth-1-ol,

4,8-dibenzyloxy-6-methylnaphth-1-ol,

4,8-dibenzyloxynaphth-1-ol,

4-benzyloxy-8-(2-chloro)ethoxynaphth-1-ol,

4-benzyloxy-8-isopropyloxynaphth-1-ol,

4-benzyloxy-8-methoxynaphth-1-ol,

4-(2,2,2-trifluoroethoxy)naphth-1-ol,

4-(2-bromo)ethoxynaphth-1-ol,

4-(2-bromo)ethoxy-5-methoxynaphth-1-ol,

4-(2-bromo)ethoxy-8-methoxynaphth-1-ol,

4-(2-chloro)ethoxynaphth-1-ol.

4-(2-chloro)ethoxy-8-methoxynaphth-1-ol,

4-(2-methoxy)ethoxynaphth-1-ol,

4-(1,4,7-trioxaheptyl)naphth-1-ol,

4-(1,4,7-trioxaoctyl)naphth-1-ol,

4-(1,4,7.10-tetraoxadecyl)naphth-1-ol,

(4-hydroxy-1-naphthyl)oxyacetic acid,

4-methoxynaphth-1-ol,

4-methoxy-5-chloronaphth-1-ol,

4-methoxy-5-chloro-8-benzyloxynaphth-1-ol,

4,8-dimethoxy-5-chloronaphth-1-ol,

4-methoxy-5-methylnaphth-1-ol.

4-methoxy-5-benzyloxynaphth-1-ol,

4-methoxy-5-benzyloxy-7-methylnaphth-1-ol,

4-methoxy-5-hydroxynaphth-1-ol,

4-methoxy-5-hydroxy-7-methylnaphth-1-ol,

4-methoxy-5-isopropyloxynaphth-1-ol,

4,5-dimethoxynaphth-1-ol,

4,5-dimethoxy-6-benzyloxynaphth-1-ol,

4,5-dimethoxy-7-methyinaphth-1-ol,

4,5-dimethoxy-8-chloronaphth-1-ol,

4-methoxy-6-methyinaphth-1-ol,

4-methoxy-6-methyl-7-acetyl-8-hydroxynaphth-1-ol,

5-4-methoxy-6,7-dimethylnaphth-1-ol,

4-methoxy-6-methyl-8-benzyloxynaphth-1-ol,

4-methoxy-6-methyl-8-hydroxynaphth-1-ol,

4,8-dimethoxy-6-methyinaphth-1-ol,

4-methoxy-6-ethoxynaphth-1-ol,

4-methoxy-6,7-diethoxynaphth-1-ol,

4-methoxy-7-methylnaphth-1-ol,

4,8-dimethoxy-7-benzyloxynaphth-1-ol,

4-methoxy-7-ethoxynaphth-1-ol,

4-methoxy-8-chloronaphth-1-ol,

4-methoxy-8-methyinaphth-1-ol,

4-methoxy-8-benzyloxynaphth-1-ol,

4-methoxy-8-hydroxynaphth-1-ol,

4-methoxy-8-isopropyloxynaphth-1-ol,

4,8-dimethoxynaphth-1-ol,

4-ethoxynaphth-1-ol,

4-propyloxynaphth-1-ol,

4-isopropyloxynaphth-1-ol,

4-butoxynaphth-1-ol,

4-isobutoxynaphth-1-ol,

4-sec-butoxynaphth-1-ol,

4-isoamoxynaphth-1-ol,

4-bis(2-chloroisopropyloxy)naphth-1-ol,

4-cyclohexyloxynaphth-1-ol,

4-octyloxynaphth-1-ol,

4-(2-chloropropoxy)naphth-1-ol,

isopropylidene-4,5-dioxynaphth-1-ol,

5-methoxynaphth-1-ol,

5,8-dimethoxy-6-methylnaphth-1-ol,

5,8-dimethoxy-6,7-dichloronaphth-1-ol,

5,8-dimethoxy-7-methyinaphth-1-ol,

5,8-diacetoxynaphth-1-ol, and

8-methoxynaphth-1-ol.

4-methoxynaphth-1-ol,

4-ethoxynaphth-1-ol,

4-isopropyloxynaphth-1-ol, and

4,8-dimethoxynaphth-1-ol

and salts thereof.

Additional oxidation dye couplers suitable for use herein include catechol species and in particular catechol “dopa” species which includes dopa itself as well as homologs, analogs and derivatives of DOPA. Other suitable dye precursors are dihydroxyindole (DHI), dihydroxyindolecarboxylic acid (DHICA) and derivatives thereof, indolines and derivatives thereof. Examples of suitable catechol species include cysteinyl dopa, alpha alkyl dopa having 1 to 4, preferably 1 to 2 carbon atoms in the alkyl group, epinephrine and dopa alkyl esters having 1 to 6, preferably 1 to 2 carbon atoms in the alkyl group.

The oxidation dye couplers (precursors) can be used herein alone or in combination with other oxidation dye couplers (precursors) mentioned above. The choice of a single dye coupler (precursor) will be determined by the color, shade and intensity of coloration which is desired. The following are preferred oxidation dye couplers (precursors) which can be used herein, singly or in combination, to provide oxidation hair dyes having a variety of shades ranging from ash blonde to black; these are: pyrogallol, resorcinol, p-toluenediamine, o-phenylenediamine, m-phenylenediamine, o-aminophenol, p-aminophenol, 4-amino-2-nitrophenol, nitro-p-phenylenediamine, N-phenyl-p-phenylenediamine, m-aminophenol, 2-amino-3-hydroxypyridine, N,N bis (2-hydroxyethyl)p-phenylenediamine, 4-amino-2-hydroxytoluene, 1,5-dihydroxynapthalene 2,4-diaminoanisole, hydroquinone, 4-amino-2-hydroxytoluene, 2-methyl resorcinol, 2-methyl-5-hydroxyaminophenol, 6-amino-3-hydroxy-toluene, 2,5-diaminotoluene, and 1-phenyl-3-methyl-pyazolone. These can be used in the molecular form or in the form of peroxide-compatible salts.

### WATER SOLUBLE CARBONATE RELEASING SALT

Compositions of the invention may also contain one or more water soluble carbonate releasing salts. Nonlimiting examples of such salts include Na₂CO₃, NaHCO₃, K₂ CO₃, KHCO₃, (NH₄)₂CO₃, NH₄HCO₃, CaCO₃ and Ca(HCO₃)₂. These compounds maybe used singly or, as required, in combination.

### WATER SOLUBLE AMMONIUM SALT

Compositions of the invention may also contain one or more water soluble ammonium salts. Nonlimiting examples of such salts include ammonium chloride, ammonium carbonate, ammonium bicarbonate, ammonium sulfate, and (or) ammonium carbamate.

Solvents

Water is the preferred principal diluent for the compositions according to the present invention. As such, the compositions of present invention may also include one or more solvents as additional diluent materials. Generally, the solvent is selected to be miscible with water and innocuous to the skin. Solvents suitable for use herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a particularly preferred group includes ethanol, isopropanol, n-propanol, butanol, propylene glycol, ethylene glycol monoethyl ether, and mixtures thereof.

The compositions of the invention may also include the following materials.

Buffering Agents

The coloring compositions of the present invention after the composition comprising one or more oxidative hair coloring agents and the composition comprising a water-soluble peroxygen oxidizing agent have been mixed together may have a pH in the range of from about 8 to about 11, more preferably from about 9 to about 11, or about 9.5 to about 11, especially from about 10 to about 11.

As herein before described the preferred coloring compositions of the present invention may contain one or more buffering agents and/or hair swelling agents (HSAs) to adjust the pH to the desired level. Several different pH modifiers can be used to adjust the pH of the final composition or any constituent part thereof.

Thickeners

The composition containing one or more oxidative hair coloring agents of the present invention (coloring compositions) may additionally include a thickener at a level of from about 0.05% to about 20%. preferably from about 0.1% to about 10%, more preferably from about 0.5% to about 5% by weight. Thickening agents suitable for use in the compositions of the invention may be selected from the group consisting of oleic acid, cetyl alcohol, oleyl alcohol, sodium chloride, cetearyl alcohol, stearyl alcohol, and synthetic thickeners; and mixtures thereof.

Surfactant Materials

The compositions of the present invention may additionally contain a surfactant system. Suitable surfactants for inclusion in the compositions of the invention generally have a lipophilic chain length of from about 8 to about 22 carbon atoms and can be selected from the group consisting of anionic, cationic, nonionic, amphoteric, zwitterionic surfactants and mixtures thereof.

Materials used in compositions of the invention may be employed in the following ranges:

EXAMPLE 1
(which shows ranges of ingredients)
Hair Coloring Composition

| Ingredients | % w/w |
|---|---|
| Oxidative dyes | 0.01-15 |
| Sequestrant | 0.01-1 |
| Antioxidant | 0.01-3 |
| Solvent | 2.0-35 |
| Buffering agent | 0.01-10 |
| Thickener | 0.05-20 |
| Surfactants | 5.0-40 |
| Water soluble carbonate releasing salt | 1.0-5.0 |
| Water soluble ammonium salt | 0-5.0 |
| Water to balance | 100 |

The following are specific nonlimiting examples of compositions of the invention which may be made. In the examples below, the pH refers to the pH after the coloring composition and the developer have been mixed.

EXAMPLE 2
Dye Formulation Ingredients

| Chemical Name | % (w/w) |
|---|---|
| p-Phenylenediamine | 0.125 |
| m-Aminophenol | 0.0058 |
| Resorcinol | 0.133 |
| Pheny-methyl-pyrazolone | 0.033 |
| N,N-bis-2-hydroxyethyl-PPD sulfate | 0.0125 |
| Sodium sulphite | 1 |
| Sodium EDTA | 0.6 |
| Sodium isoascorbate | 0.15 |
| Propylene glycol | 8.6 |
| Oleic acid (5 Titre) | 8.6 |
| Isopropanol | 12.5 |
| Perfume oil | 0.5 |
| Dihydroxyethyl soyamine dioleate | 22.2 |
| PEG3 Cocamine | 8 |
| Ammonium carbonate | 6 |
| NaOH to pH10 | |
| Water to balance | 100 |

EXAMPLE 3
Dye Formulation Ingredients

| Chemical Name | % (w/w) |
|---|---|
| p-Phenylenediamine | 1 |
| 4-Amino-2-hydroxytoluene | 1 |
| Sodium sulphite | 1 |
| Sodium EDTA | 0.6 |
| Sodium isoascorbate | 0.15 |
| Propylene glycol | 12 |
| Oleic acid (5 Titre) | 8.6 |
| Isopropanol | 12.5 |
| Perfume oil | 0.5 |
| Dihydroxyethyl soyamine dioleate | 22.2 |
| PEG3 Cocamine | 8 |
| Ammonium carbonate | 6 |
| NaOH to pH10 | |
| Water to balance | 100 |

EXAMPLE 4
Dye Formulation Ingredients

| Chemical Name | % (w/w) |
|---|---|
| p-Phenylenediamine | 1 |
| Resorcinol | 1 |
| Sodium sulphite | 1 |
| Sodkim EDTA | 0.6 |
| Sodium isoascorbate | 0.15 |
| Propylene glycol | 12 |
| Oleic acid (5 Titre) | 8.6 |
| Isopropanol | 12.5 |
| Perfume oil | 0.5 |
| Dihydroxyethyl soyamine dioleate | 22.2 |
| PEG3 Cocamine | 8 |
| Sodium bicarbonate | 3 |
| Ammonium chloride | 3 |
| NaOH to Ph10 | |
| Water to balance | 100 |

EXAMPLE 5
Dye Formulation Ingredients

| Chemical Name | % (w/w) |
|---|---|
| p-Phenylenediamine | 1 |
| Resorcinol | 1 |
| Sodium sulphite | 1 |
| Sodium EDTA | 0.6 |
| Sodium isoascorbate | 0.15 |
| Propylene glycol | 12 |
| Oleic acid (5 Titre) | 8.6 |
| Isopropanol | 12.5 |
| Perfume oil | 0.5 |
| Dihydroxyethyl soyamine dioleate | 22.2 |
| PEG3 Cocamine | 8 |
| Ammonium carbonate | 6 |
| NaOH to pH10 | |
| Water to balance | 100 |

EXAMPLE 6
Dye Formulation Ingredients

| Chemical Name | % (w/w) |
|---|---|
| p-Aminophenol | 0.4 |
| 4-Amino-2-hydroxytoluene | 0.4 |
| Sodium sulphite | 1 |
| Sodium EDTA | 0.6 |
| Sodium isoascorbate | 0.15 |
| Propylene glycol | 12 |
| Oleic acid (5 Titre) | 8.6 |
| Isopropanol | 12.5 |
| Perfume oil | 0.5 |
| Dihydroxyethyl soyamine dioleate | 22.2 |
| PEG3 Cocamine | 8 |
| Ammonium carbonate | 6 |
| NaOH to pH10 | |
| Water to balance | 100 |

EXAMPLE 7
Developer: Formulation Ingredients

| Chemical Name | % (w/w) |
|---|---|
| Ceteareth-7 | 1.00 |
| Polyquaternium-37 | 1.00 |
| 50% Hydrogen Peroxide | 12.00 |
| 85% Phosphoric Acid | 0.03% |
| Water to balance | q.s. |
| | 100 |

EXAMPLE 8
(which shows ranges of ingredients)
Developer Formulation or Composition
Containing Water-Soluble Peroxygen Oxidizing Agent

| Chemical Name | % (w/w) |
|---|---|
| Sequestrant | 0.01-1 |
| Antioxidant | 0.01-3 |
| Solvent | 2.0-35 |
| Buffering agent | 0.01-10 |
| Thickener | 0.05-20 |
| Surfactants | 5.0-40 |
| Oxidizing agent | 0.01-9 |
| Water to balance | 100 |

EXAMPLE 9—
Developer Formulation

| | % w/w |
|---|---|
| Oxidizing agent | 0.01-9 |
| Water to balance | to |
| | 100 |

PREPARATION OF THE DYE COMPOSITION—that is, the composition which comprises oxidative hair coloring agents:

Surfactants, perfume oil and solvent are mixed at 55° C. to obtain a homogenous solution (part 1). Deionized water is added to the beaker and mixing is continued. Antioxidants are added followed by solvent and nitrogen blanketing is begun. Dyes are added and the mixture is heated to 50-55° C. and further mixed until the solution is clear. The solution is cooled to 20-35° C. The solution pH is then adjusted to 9-12 with either concentrated ammonium hydroxide and/or 50% sodium hydroxide. Thereupon at least one water soluble carbonate releasing salt and optionally a water soluble ammonium salt is added. After salt addition is complete the pH is again adjusted to be in the range 9.5-11. Optionally more water can be added.

### PREPARATION OF WATER-SOLUBLE PEROXYGEN OXIDIZING AGENT FORMULATION

Add deionized water to beaker and begin mixing. Add surfactants, thickener and buffering agents follow by oxidizing agent and remaining ingredients to water.

### HOW TO USE COMPOSITIONS OF THE INVENTION

A mixture of the following two compositions is made just prior to application to the hair.

(a) a composition comprising a water-soluble peroxygen oxidizing agent; and

(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises al least one water soluble carbonate releasing salts; and optionally a water soluble ammonium salt.

The hair to be colored can first be made wet with water. Application temperatures may be in the range from 15 to 40 degrees C. Then a water-soluble peroxygen oxidizing agent; and one or more oxidative hair coloring agents as described above, are thoroughly mixed together, and soon after, this mixture is applied to the hair. Again application temperatures may be in the range from 15 to 40 degrees C. After a contact time of about 2 to about 60 minutes preferably about 5 to about 30 minutes, the hair is thoroughly rinsed.

While the invention has been described in connection with preferred embodiments, this description is not intended to limit the invention to the particular embodiments set forth. To the contrary, this description is intended to cover such alternatives, modifications, and equivalents as may be included within the spirit and scope of the invention as defined by the appended claims.

The hair color lifting properties of the compositions of the present invention can be demonstrated by the following test. To a weighed swatch of Caucasian dark brown hair whose L a b components had been measured is added two parts by weight of a 1:2 mixture of composition A and a modified composition B. Composition B is modified for the color lifting test by removing the oxidative hair coloring agents. The mixture is intimately worked through the hair swatch over the period of 30 minutes. At the end of this period the hair swatch is rinsed with water, air-dried and the L a b values measured. The difference in L a b before and after treatment indicates the degree of hair color lifting.

The hair color properties of the compositions of the present invention can be demonstrated by the following test. To a weighed swatch of Piedmont natural white hair whose L a b components had been measured is added two parts by weight of a 1:2 mixture of composition A and composition B as described above. The mixture is intimately worked through the hair swatch over the period of 30 minutes. At the end of this period the hair swatch is rinsed with water, air-dried and the L a b values measured. The change in hair color (ΔE) is determined by the square root of the squares of the differences in L a b before and after treatment.

What is claimed is:

1. A composition for coloring hair which comprises a composition comprising:
the following two compositions which are mixed just prior to application to the hair:
(a) a composition comprising a water-soluble peroxygen oxidizing agent; and
(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises al least one water soluble carbonate releasing salts; and optionally a water soluble ammonium salt.

2. A composition according to claim 1, wherein the water-soluble peroxygen oxidizing agent is selected from the group consisting of hydrogen peroxide, sodium perforate, sodium percarbonate, and urea peroxide complexes.

3. A composition according to claim 1, which comprises one or more oxidative hair coloring agents which further comprises a buffering agent.

4. A composition according to claim 1, which has a pH of about 9 to about 11.

5. A composition according to claim 1, which further comprises a surfactant selected from the group consisting of anionic, nonionic, cationic, zwitterionic, amphoteric surfactants and mixtures thereof.

6. A composition according to claim 1 wherein the water soluble carbonate releasing salt is selected from the group consisting of Na₂CO₃, NaHCO₃, K₂ CO₃, KHCO₃, (NH₄)₂ CO₃, NH₄HCO₃, CaCO₃, Ca(HCO₃)₂ and mixtures thereof.

7. A composition according to claim 1 wherein the water soluble ammonium salt is selected from the group consisting of ammonium chloride, ammonium carbonate, ammonium bicarbonate, ammonium sulfate, ammonium carbamate, and mixtures thereof.

8. A composition according to claim 1 which comprises:

| Ingredients | % w/w |
|---|---|
| Oxidative dyes | 0.01-15 |
| Sequestrant | 0.01-1 |
| Antioxidant | 0.01-3 |
| Solvent | 2.0-35 |
| Buffering agent | 0.01-10 |
| Thickener | 0.05-20 |
| Surfactants | 5.0-40 |
| Water soluble carbonate releasing salt | 1.0-5.0 |
| Water soluble ammonium salt | 0-5.0 |
| Water to | 100 |

9. A process for coloring human or animal hair which comprises applying to said hair a composition comprising the following two compositions which are mixed just prior to said second application to the hair:
(a) a composition comprising from about 0.5% to about 20% by weight of a water-soluble peroxygen oxidizing agent; and
(b) a composition comprising one or more oxidative hair coloring agents selected from the group consisting of an aromatic diamine, an amino phenol, a naphthol, a polyhydric phenol, a catechol and mixtures thereof; wherein the composition comprising one or more oxidative hair coloring agents further comprises al least one water soluble carbonate releasing salts; and optionally a water soluble ammonium salt. wherein the composition comprising one or more oxidative hair coloring agents has a pH of from about 9 to about 11.

10. A hair coloring kit comprising an individually packaged oxidizing component, an individually packaged component having one or more hair coloring agents, a component wherein said components are compositions are in accordance with claim 1.
